(19) United States
(12) Patent Application Publication
(10) Pub. No.: US 2022/0304637 A1
(43) Pub. Date: Sep. 29, 2022

(54) INTRA-ORAL X-RAY IMAGING SYSTEM HAVING CHARGING DECK

(71) Applicant: NANORAY CO., LTD., Daegu (KR)

(72) Inventors: Jae Yoon PARK, Gyeonggi-do (KR); Won SEONG, Daejeon (KR)

(21) Appl. No.: 17/413,994

(22) PCT Filed: Apr. 16, 2020
(86) PCT No.: PCT/KR2020/005117

§ 371 Date: Jun. 15, 2021

(51) Int. Cl. A61B 6/14; A61B 6/00; G03B 42/04

(52) U.S. Cl. CPC A61B 6/145; A61B 6/4208; G03B 42/042; A61B 6/4429

(57) ABSTRACT

An intra-oral X-ray imaging system according to an embodiment of the present disclosure includes an intra-oral X-ray sensing device being shaped in such a manner as to be hand-gripped by an user and performing X-ray imaging within an oral cavity through X-ray radiation, a charging connection deck having a structure where the intra-oral X-ray sensing device is accommodated therein and is chargeable, the charging connection deck being electrically connected to the intra-oral X-ray sensing device when the intra-oral X-ray sensing device is accommodated, and an X-ray image generation and display device being electrically connected to the charging connection deck, image-processing X-ray data resulting from the X-ray imaging, generating an X-ray image as a result of the image-processing, and displaying the generated X-ray image for a user's viewing, wherein the X-ray image generation and display device receives the X-ray data resulting from the X-ray imaging, through the charging connection deck.

### CROSS REFERENCE TO RELATED APPLICATIONS AND CLAIM OF PRIORITY

This application claims benefit under 35 U.S.C. 119(e), 120, 121, or 365(c), and is a National Stage entry from International Application No. PCT/KR2020/005117, filed Apr. 16, 2020, the entire contents of which are incorporated herein by reference.

### BACKGROUND

### 1. Technical Field

The present invention relates to an intra-oral X-ray imaging system and, more particularly, to an intra-oral X-ray imaging system having a charging deck, the imaging system including a charging connection deck on which an intra-oral X-ray sensing device is placed and in which an X-ray image resulting from imaging by the intra-oral X-ray sensing device is displayed for a user's viewing on an image display device through the charging connection deck.

### 2. Background Art

An intra-oral X-ray sensor is a sensor that is inserted into an oral cavity at a dental clinic and is used to obtain an X-ray image with X-ray radiation emitted from the outside.

The intra-oral X-ray sensor is mostly used when there is a need to obtain an X-ray image of a surface of a narrow area of an oral cavity. FIG. 1 is a view illustrating a configuration of a commonly used X-ray sensor positioned within the oral cavity.

As illustrated in FIG. 1, the X-ray sensor positioned within the oral cavity has a structure where a rectangle-shaped X-ray sensor is connected to a strand of electric wire cable. A dentist at a dental clinic places the X-ray sensor into a patient's oral cavity and then positions the X-ray sensor close to a tooth of which an image needs to be captured. Then, an external X-ray radiation apparatus emits X-rays in a state where the patient holds the X-ray sensor with his/her hand and keeps the X-ray sensor stationary in order for the X-ray sensor not to move away from the position thereof. In this manner, the X-ray image is obtained.

However, in the case of the X-ray sensor positioned within the oral cavity in the related art, the dentist inserts his/her fingers into the patient's oral cavity and then moves the X-ray sensor to a desired position with his/her fingers. This practice is unsanitary because bacteria on the dentist's fingers may spread to a patient's body through the patient's oral cavity.

In addition, in a case where X-ray imaging is performed within the oral cavity at the dental clinic, after performing the X-ray imaging of a patient's area whose image needs be obtained, in order to view an X-ray image, the dentist is required to mount the X-ray sensor on an X-ray sensor mounting unit or socket connected to a computer on which a program for generating the X-ray image is installed. Then, the dentist is required to view the X-ray image resulting from the X-ray imaging by the X-ray sensor by performing a corresponding operation on the computer. That is, the dentist cannot immediately view the X-ray image at a place where the X-ray imaging is performed and thus is required to move to a specific place to view the X-ray image. Thus, it is very inconvenient to view the X-ray image.

In addition, in the related art, after performing the X-ray imaging, in order to view the X-ray image, the dentist is required to remove the X-ray image sensor from a sensing device, to insert the removed X-ray image sensor into the X-ray sensor mounting unit or socket connected to the computer, and to perform processing on the loaded corresponding file on the computer, using an associated program. These jobs are bothersome.

### SUMMARY

An objective of the present invention, which is made to solve the above-described problems, is to provide an intra-oral X-ray imaging system capable of automatically performing image processing and displaying an X-ray image only when an intra-oral X-ray sensing device is placed on a charging deck that is configured to charge the intra-oral X-ray sensing device with electricity. In the intra-oral X-ray imaging system, the X-ray image is very easy to view after performing X-ray imaging.

According to an aspect of the present invention, there is provided an intra-oral X-ray imaging system having a charging deck, the intra-oral X-ray imaging system including: an intra-oral X-ray sensing device being shaped in such a manner as to be hand-gripped by an user and performing X-ray imaging within an oral cavity through X-ray radiation; a charging connection deck having a structure where the intra-oral X-ray sensing device is accommodated therein and is chargeable, the charging connection deck being electrically connected to the intra-oral X-ray sensing device when the intra-oral X-ray sensing device is accommodated; and an X-ray image generation and display device being electrically connected to the charging connection deck, image-processing X-ray data resulting from the X-ray imaging by the intra-oral X-ray sensing device, generating an X-ray image as a result of the image-processing, and displaying the generated X-ray image for a user's viewing, wherein the X-ray image generation and display device receives the X-ray data resulting from the X-ray imaging by the intra-oral X-ray sensing device, through the charging connection deck.

In the intra-oral X-ray imaging system, when the intra-oral X-ray sensing device is accommodated in the charging connection deck and is electrically connected thereto, the X-ray image generation and display device may automatically receive the X-ray data resulting from the X-ray imaging by the intra-oral X-ray sensing device, may image-process the received X-ray data, may generate an X-ray image as a result of the image-processing, and may display the generated X-ray image for the user's viewing.

According to another aspect of the present invention, there is provided an intra-oral X-ray imaging system including: an intra-oral X-ray sensing device being shaped in such a manner as to be hand-gripped by a user and performing X-ray imaging within an oral cavity through X-ray radiation; and a charging connection deck device having a structure where the intra-oral X-ray sensing device is accommodated therein and is chargeable, the charging connection deck device being electrically connected to the intra-oral X-ray sensing device when the intra-oral X-ray sensing device is accommodated, wherein the charging connection deck device including: a charging deck unit charging the intra-oral X-ray sensing device; an X-ray image generation unit image-processing X-ray data resulting from the X-ray imaging by the intra-oral X-ray sensing device and generating an X-ray image as a result of the image-processing; and an X-ray image display unit displaying the X-ray image generated by the X-ray image generation unit for a user's viewing.

In the intra-oral X-ray imaging system, when the intra-oral X-ray sensing device is accommodated in the charging deck unit and is electrically connected thereto, the X-ray image generation unit may automatically receive the X-ray data resulting from the X-ray imaging by the intra-oral X-ray sensing device, may image-process the received X-ray data, generates an X-ray image as a result of the image-processing, and may transfer the generated X-ray image to the X-ray image display unit.

In the intra-oral X-ray imaging system, the intra-oral X-ray sensing device may include: an intra-oral X-ray sensor unit performing X-ray imaging within the oral cavity through by the X-ray radiation; and a handle being shaped as a long bar in such a manner as to be hand-gripped by the user and being combined with the X-ray sensor unit.

In the intra-oral X-ray imaging system, the handle may include: a hand-gripped portion being hand-gripped by the user; and an extension arm being connected to the X-ray sensing unit.

In the intra-oral X-ray imaging system, the intra-oral X-ray sensing device may include: a battery supplying power to the X-ray sensing unit; and a battery charging unit charging the battery.

According to the present invention, in order to view an X-ray image acquired by a hand-gripped intra-oral X-ray sensing device, there is no need to electrically connect the hand-gripped oral-cavity X-Ray sensing device to a separately installed X-ray image generation and display device, or to separate a removably built-in memory device from the hand-gripped intra-oral X-ray sensing device and connect the removed memory device to the X-ray image generation and display device. The X-ray image generation and display device performs processing on X-ray image data, and thus the X-ray image can be viewed only when the hand-gripped intra-oral X-ray sensing device is placed in the upright position in a charging connection deck positioned in the vicinity of the hand-gripped intra-oral X-ray sensing device. Thus, there is an advantage in that it is very convenient for a user to view the X-ray image.

In addition, according to the present invention, the charging connection deck performs a function of a charging device configured to charge the hand-gripped intra-oral X-ray sensing device with electricity. Thus, for charging, there is no need to connect the hand-gripped intra-oral X-ray sensing device to a separate device. A battery built into the hand-gripped intra-oral X-ray sensing device is charged only when the hand-gripped intra-oral X-ray sensing device is placed in the upright position in the charging connection deck. Thus, there is an advantage in that the battery is very conveniently charged with electricity.

### BRIEF DESCRIPTION OF THE DRAWINGS

FIG. 1 is a view illustrating a configuration of an intra-oral X-ray sensor in the related art;

FIG. 2 is a view illustrating a configuration of an intra-oral X-ray imaging system according to a first embodiment of the present invention;

FIG. 3 is a view illustrating a state where an intra-oral X-ray sensor is placed on a charging connection deck in the intra-oral X-ray imaging system according to the first embodiment of the present invention;

FIG. 4 is a view illustrating the state where the intra-oral X-ray sensor is placed on the charging connection deck in the intra-oral X-ray imaging system according to the first embodiment of the present invention;

FIG. 5 is a view illustrating a state where an intra-oral X-ray sensor is placed on a charging connection deck device of an intra-oral X-ray imaging system according to a second embodiment of the present invention;

FIG. 6 is a view illustrating the state where the intra-oral X-ray sensor is placed on the charging connection deck device of the intra-oral X-ray imaging system according to the second embodiment of the present invention;

FIG. 7 is a view illustrating a configuration of an example of an oral cavity X-ray sensing device of the intra-oral X-ray imaging system according to the second embodiment of the present invention; and

FIG. 8 is a view illustrating a configuration of another example of the intra-oral X-ray sensing device of the intra-oral X-ray imaging system according to the second embodiment of the present invention.

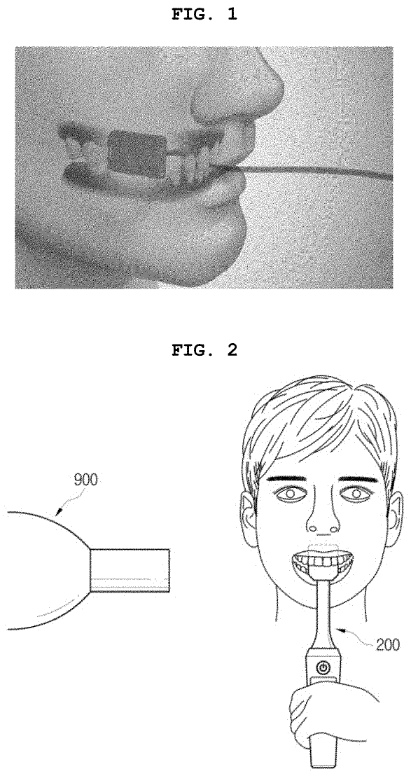

### DETAILED DESCRIPTION

An intra-oral X-ray imaging system will be described. The intra-oral X-ray imaging system is configured to include an intra-oral X-ray sensing device, a charging connection deck, and an X-ray image generation and display device. The intra-oral X-ray sensing device is shaped in such a manner as to be hand-gripped by a user and performs X-ray imaging within an oral cavity through X-ray radiation.

The charging connection deck has a structure where the intra-oral X-ray sensing device is accommodated therein and is chargeable and, when the intra-oral X-ray sensing device is accommodated therein, is electrically connected to the intra-oral X-ray sensing device.

The X-ray image generation and display device is electrically connected to the charging connection deck, image-processes X-ray data resulting from the X-ray imaging by the intra-oral X-ray sensing device, generates an X-ray image as a result of the image-processing, and displays the generated X-ray image for a user's viewing.

The X-ray image generation and display device receives the X-ray data resulting from the X-ray imaging by the intra-oral X-ray sensing device, through the charging connection deck.

### First Embodiment

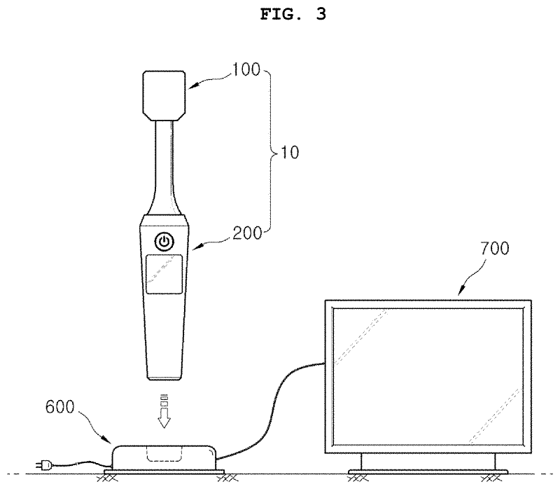

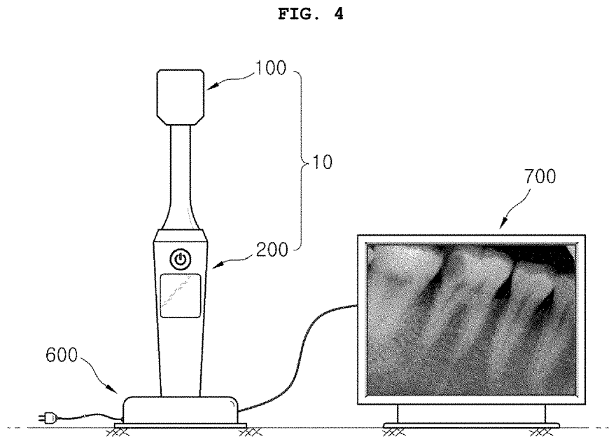

FIG. 2 is a view illustrating a configuration of an intra-oral X-ray imaging system according to a first embodiment of the present invention. FIG. 3 is a view illustrating a state where an intra-oral X-ray sensor is placed on a charging connection deck in the intra-oral X-ray imaging system according to the first embodiment of the present invention. FIG. 4 is a view illustrating the state where the intra-oral X-ray sensor is placed on the charging connection deck in the intra-oral X-ray imaging system according to the first embodiment of the present invention.

With reference to the drawings, an intra-oral X-ray imaging system having a charging deck according to the first embodiment of the present invention is configured to include an intra-oral X-ray sensing device 10 including an X-ray sensing unit 100 and a handle 200, a charging connection deck 600, and an X-ray image generation and display device 700.

The X-ray sensing unit 100 performs X-ray imaging within the oral cavity to obtain an X-ray image through the X-ray radiation from the outside. When X-rays occur in an X-ray tube 900 for the X-ray radiation and are emitted therefrom, the X-ray sensing unit 100 captures an X-ray image of a patient's imaging area.

The handle 200 is shaped in the form of a long bar in such a manner as to fix the X-ray sensing unit 100 and to be hand-gripped by the user.

The charging connection deck 600 is shaped in such a manner as to accommodate the handle 200. The charging connection deck 600 is electrically connected to the handle 200. Through the handle 200, the charging connection deck 600 supplies power to the intra-oral X-ray sensing device 10 and charges the intra-oral X-ray sensing device 10. Alternatively, the charging connection deck 600 transfers the X-ray data from the intra-oral X-ray sensing device to the X-ray image generation and display device 700.

The X-ray image generation and display device 700 is electrically connected to the charging connection deck 600 and image-processes the X-ray data resulting from the X-ray imaging by the intra-oral X-ray sensing device 10, using an X-ray image processing program. Then, the X-ray image generation and display device 700 generates an X-ray image as a result of the image-processing and displays the generated X-ray image for the user's viewing. The X-ray image processing program is a program for image-processing the X-ray data acquired from the intra-oral X-ray sensing device 10 and creating an X-ray image file in the form that the user can recognize with unaided vision. The X-ray image processing program is well known to a person of ordinary skill in the art to which the present invention pertains, and thus a detailed description thereof is omitted.

The X-ray image generation and display device 700 may be realized as a personal computer or a server computer equipped with a monitor.

The X-ray image generation and display device 700 has a sensor connection detection function of detecting whether or not the intra-oral X-ray sensing device 10 is connected to the charging connection deck 600. Therefore, when the intra-oral X-ray sensing device 10 is connected to the charging connection deck 600, the X-ray image generation and display device 700 detects this connection. Then, the X-ray image generation and display device 700 has access to X-ray data stored in the intra-oral X-ray sensing device 10, receives the stored X-ray data, generates an X-ray image using the stored X-ray data, and displays the generated X-ray image.

When the intra-oral X-ray sensing device 10 is connected to the charging connection deck 600, the X-ray image generation and display device 700 automatically detects this connection using the sensor connection detection function without the user performing any other operation. Then, the X-ray image generation and display device 700 generates the X-ray image using the X-ray data stored in the intra-oral X-ray sensing device 10 and then displays the generated X-ray image on the X-ray image generation and display device 700 for the user's viewing. Therefore, after performing X-ray imaging using the intra-oral X-ray sensing device 10, the user only needs to place the intra-oral X-ray sensing device 10 on the charging connection deck 600. Then, the user can recognize the X-ray image displayed automatically on the X-ray image generation and display device 700 without performing any other operation.

In addition, the charging connection deck 600 is also used to charge the intra-oral X-ray sensing device 10. The intra-oral X-ray sensing device 10 includes a battery supplying power to the X-ray sensing unit 100, the X-ray image generation and display device 700, and the like, and a battery charging unit charging the battery. Thus, with this configuration, the battery does not need to be replaced each time energy thereof is used up and can be re-used only by being mounted on the charging connection deck 600 and thus being charged.

In a case where the battery is mounted on the charging connection deck 600 for charging, the battery is charged when the intra-oral X-ray sensing device 10 is placed in the upright position in the charging connection deck 600 in a state where the battery is built into the intra-oral X-ray sensing device 10 without being separated from the intra-oral X-ray sensing device 10.

In addition, the hand-gripped intra-oral X-ray sensing device 10 according to the first embodiment of the present invention includes a memory unit in which the captured X-ray image is stored.

### Second Embodiment

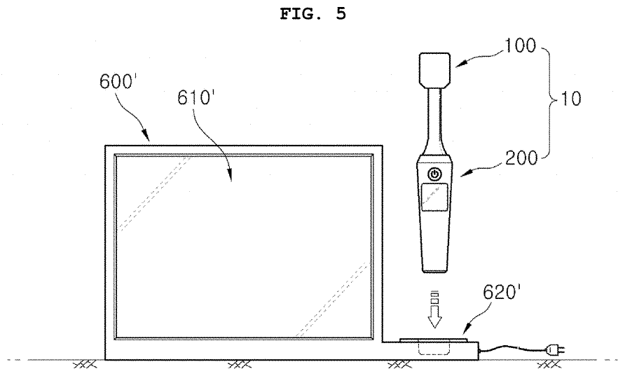

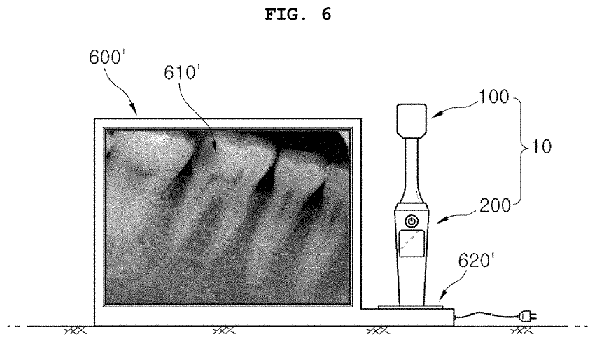

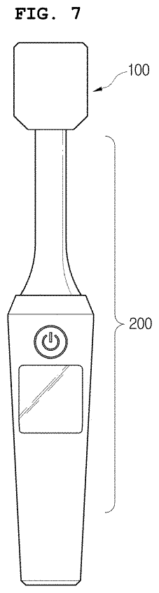

FIG. 5 is a view illustrating a state where an intra-oral X-ray sensor is placed on a charging connection deck device of an intra-oral X-ray imaging system according to a second embodiment of the present invention. FIG. 6 is a view illustrating the state where the intra-oral X-ray sensor is placed on the charging connection deck device of the intra-oral X-ray imaging system according to the second embodiment of the present invention.

An intra-oral X-ray imaging system having a charging deck according to the second embodiment of the present invention is configured to include the intra-oral X-ray sensing device 10 and a charging connection deck device 600′.

The intra-oral X-ray sensing device 10 is shaped in such a manner as to be hand-gripped by the user and performs the X-ray imaging within the oral cavity through the X-ray radiation.

The charging connection deck device 600′ has a structure where the intra-oral X-ray sensing device 10 is accommodated therein and is chargeable and, when the intra-oral X-ray sensing device 10 is accommodated therein, is electrically connected to the intra-oral X-ray sensing device 10.

The charging connection deck device 600′ is configured to include a charging deck unit 620′, an X-ray image generation unit (not illustrated) and an X-ray image display unit 610′.

The charging deck unit 620′ charges the intra-oral X-ray sensing device 10.

The X-ray image generation unit image-processes the X-ray data resulting from the X-ray imaging by the intra-oral X-ray sensing device 10 and generates the X-ray image as a result of the image-processing.

The X-ray image generation unit image-processes the X-ray data resulting from the X-ray imaging by the X-ray sensing unit 100 using the X-ray image processing program, generates the X-ray image as a result of the image-processing, and displays the generated X-ray image for the user's viewing. The X-ray image processing program is a program for image-processing the X-ray data acquired from the intra-oral X-ray sensing device 10 and creating an X-ray image file in the form that the user can recognize with unaided vision. The X-ray image processing program is well known to a person of ordinary skill in the art to which the present invention pertains, and thus a detailed description thereof is omitted.

The X-ray image display unit 610′ displays the X-ray image generated by the X-ray image generation unit for the user's viewing.

When the intra-oral X-ray sensing device 10 is accommodated in the charging deck unit 620′ and is electrically connected thereto, the X-ray image generation unit automatically receives the X-ray data resulting from the X-ray imaging by the intra-oral X-ray sensing device 10, image-process the received X-ray data, generates the X-ray image as a result of the image-processing, and transfers the generated X-ray image to the X-ray image display unit 610′.

FIG. 7 is a view illustrating a configuration of an example of the intra-oral X-ray sensing device 10 of the oral cavity X-ray imaging system according to the second embodiment of the present invention.

With reference to FIG. 7, according to the second embodiment of the present invention, the intra-oral X-ray sensing device 10 is configured to have a structure where the handle 200 and the X-ray sensing unit 100 are combined with each other by connecting a sensor combination portion positioned on one end portion of the handle 200 and the X-ray sensing unit 100 together as if with a hinge.

The sensor combination portion is disc-shaped. A disc-shaped hole in which the disc-shaped sensor combination portion is nested is formed in the X-ray sensing unit 100. The disc-shaped sensor combination portion is inserted into the disc-shaped hole, and thus the X-ray sensing unit 100 is rotatable with respect to the handle 200. That is, with this structure providing the same function as a hinge, the X-ray sensing unit 100 is rotatable in the leftward-rightward direction with respect to the handle 200.

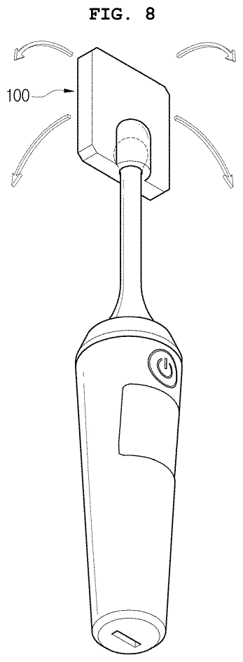

FIG. 8 is a view illustrating a configuration of another example of the intra-oral X-ray sensing device 10 of the oral cavity imaging system according to the second embodiment of the present invention.

With reference to FIG. 8, in the intra-oral X-ray sensing device 10 according to the second embodiment of the present invention, the sensor combination portion positioned on one end portion of the handle 200 and the X-ray sensing unit 100 are combined with each other by employing a ball-joint structure. Thus, the X-ray sensing unit 100 is capable of undergoing a rotational motion with respect to the handle 200, such as pitching, rolling, or yawing. The sensor combination portion is provided in the form of a ball stud for a ball joint on one side of an extension arm 210. A bearing portion to be combined with the ball stud is provided on a surface, opposite in direction to a surface on which a sensor is arranged, of the X-ray sensing unit 100. With this configuration, the ball stud and the bearing portion are combined with each other. Thus, the X-ray sensing unit 100 is capable of undergoing a rotational motion with respect to the sensor combination portion, such as yawing.

The intra-oral X-ray imaging system having a charging deck according to the embodiment of the present invention can be used in the medical field in which there is a need to perform X-ray imaging within a patient's oral cavity.

1: An intra-oral X-ray imaging system comprising:
an intra-oral X-ray sensing device being shaped in such a manner as to be hand-gripped by a user and being configured to perform X-ray imaging within an oral cavity through X-ray radiation;
a charging connection deck having a structure where the intra-oral X-ray sensing device is accommodated therein and is chargeable, the charging connection deck being configured to be electrically connected to the intra-oral X-ray sensing device when the intra-oral X-ray sensing device is accommodated; and
an X-ray image generation and display device being configured to be electrically connected to the charging connection deck, image-process X-ray data resulting from the X-ray imaging by the intra-oral X-ray sensing device, generate an X-ray image as a result of the image-processing, and display the generated X-ray image for a user's viewing,
wherein the X-ray image generation and display device receives the X-ray data resulting from the X-ray imaging by the intra-oral X-ray sensing device, through the charging connection deck.

2: The intra-oral X-ray imaging system of claim 1, wherein when the intra-oral X-ray sensing device is accommodated in the charging connection deck and is electrically connected thereto, the X-ray image generation and display device automatically receives the X-ray data resulting from the X-ray imaging by the intra-oral X-ray sensing device, image-processes the received X-ray data, generates the X-ray image as a result of the image-processing, and displays the generated X-ray image for the user's viewing.

3: An intra-oral X-ray imaging system comprising:
an intra-oral X-ray sensing device being shaped in such a manner as to be hand-gripped by a user and being configured to perform X-ray imaging within an oral cavity through X-ray radiation; and
a charging connection deck device having a structure where the intra-oral X-ray sensing device is accommodated therein and is chargeable, the charging connection deck device being configured to be electrically connected to the intra-oral X-ray sensing device when the intra-oral X-ray sensing device is accommodated, the charging connection deck device comprising:
a charging deck unit being configured to charge the intra-oral X-ray sensing device with electricity;
an X-ray image generation unit being configured to image-process X-ray data resulting from the X-ray imaging by the intra-oral X-ray sensing device and generate an X-ray image as a result of the image-processing; and
an X-ray image display unit being configured to display the X-ray image generated by the X-ray image generation unit for a user's viewing.

4: The intra-oral X-ray imaging system of claim 1, wherein when the intra-oral X-ray sensing device is accommodated in the charging deck unit and is electrically connected thereto, the X-ray image generation unit automatically receives the X-ray data resulting from the X-ray imaging by the intra-oral X-ray sensing device, image-processes the received X-ray data, generates an X-ray image as a result of the image-processing, and transfers the generated X-ray image to the X-ray image display unit.

5: The intra-oral X-ray imaging system of claim 1, wherein the intra-oral X-ray sensing device comprises:
an intra-oral X-ray sensor unit being configured to perform X-ray imaging within the oral cavity through the X-ray radiation; and
a handle being shaped as a long bar in such a manner as to be hand-gripped by the user and being combined with the X-ray sensor unit.

6: The intra-oral X-ray imaging system of claim 5, wherein the handle comprises:
a hand-gripped portion being configured to be hand-gripped by the user; and
an extension arm being configured to be connected to the X-ray sensing unit.

7: The intra-oral X-ray imaging system of claim 5, wherein the intra-oral X-ray sensing device comprises:
a battery configured to supply power to the X-ray sensing unit; and
a battery charging unit configured to charge the battery with electricity.

8: The intra-oral X-ray imaging system of claim 2, wherein the intra-oral X-ray sensing device comprises:
an intra-oral X-ray sensor unit being configured to perform X-ray imaging within the oral cavity through the X-ray radiation; and
a handle being shaped as a long bar in such a manner as to be hand-gripped by the user and being combined with the X-ray sensor unit.

9: The intra-oral X-ray imaging system of claim 8, wherein the handle comprises:
a hand-gripped portion being configured to be hand-gripped by the user; and
an extension arm being configured to be connected to the X-ray sensing unit.

10: The intra-oral X-ray imaging system of claim 8, wherein the intra-oral X-ray sensing device comprises:
a battery configured to supply power to the X-ray sensing unit; and
a battery charging unit configured to charge the battery with electricity.

11: The intra-oral X-ray imaging system of claim 3, wherein the intra-oral X-ray sensing device comprises:
an intra-oral X-ray sensor unit being configured to perform X-ray imaging within the oral cavity through the X-ray radiation; and
a handle being shaped as a long bar in such a manner as to be hand-gripped by the user and being combined with the X-ray sensor unit.

12: The intra-oral X-ray imaging system of claim 11, wherein the handle comprises:
a hand-gripped portion being configured to be hand-gripped by the user; and
an extension arm being configured to be connected to the X-ray sensing unit.

13: The intra-oral X-ray imaging system of claim 11, wherein the intra-oral X-ray sensing device comprises:
a battery configured to supply power to the X-ray sensing unit; and
a battery charging unit configured to charge the battery with electricity.

14: The intra-oral X-ray imaging system of claim 4, wherein the intra-oral X-ray sensing device comprises:
an intra-oral X-ray sensor unit being configured to perform X-ray imaging within the oral cavity through the X-ray radiation; and
a handle being shaped as a long bar in such a manner as to be hand-gripped by the user and being combined with the X-ray sensor unit.

15: The intra-oral X-ray imaging system of claim 14, wherein the handle comprises:
a hand-gripped portion being configured to be hand-gripped by the user; and
an extension arm being configured to be connected to the X-ray sensing unit.

16: The intra-oral X-ray imaging system of claim 14, wherein the intra-oral X-ray sensing device comprises:
a battery configured to supply power to the X-ray sensing unit; and
a battery charging unit configured to charge the battery with electricity.
